(19) United States
(12) Patent Application Publication
(10) Pub. No.: US 2003/0187330 A1
(43) Pub. Date: Oct. 2, 2003

(54) Electronic endoscope apparatus using micromirror device

(75) Inventor: Kazunori Abe, Saitama-shi (JP)

Correspondence Address: Ronald R. Snider, Snider & Associates, P.O. Box 27613, Washington, DC, 20038-7613, US

(73) Assignee: Fuji Photo Optical Co., Ltd., Saitama-shi (JP)

(21) Appl. No.: 10/385,687

(22) Filed: Mar. 12, 2003

(30) Foreign Application Priority Data
Mar. 28, 2002 (JP) 2002-92962
Mar. 28, 2002 (JP) 2002-92961

(51) Int. Cl. A61B 1/06

(52) U.S. Cl. 600/180; 600/178

(57) ABSTRACT

In an electronic endoscope apparatus according to the present invention, a micromirror device reflects light from a light source to allow it to enter a light guide. A light blocking period is set by driving the polarization angles of micromirrors of the micromirror device to a light guide non-incident angle θs. This light blocking period is utilized to read signals for all pixels for one frame stored in a CCD by the same exposure. Thus, the light blocking period can be promptly set to obtain a stably bright motion picture not affected by any motions, or the like. Further, the quantity of light can be controlled by controlling the polarization angles in the micromirror device. Furthermore, desired white light is obtained.

### BACKGROUND OF THE INVENTION

This application claims the priority of Japanese Patent Applications Nos. 2002-92962 and 2002-92961 filed on Mar. 28, 2002 which are incorporated herein by reference.

1. Field of the Invention

The present invention relates to an electric endoscope apparatus that uses a light guide to guide light from a light source to a leading portion of an endoscope, the electronic endoscope apparatus having an arrangement that controls the quantity of light emitted from the leading portion and that sets a light blocking period used to read signals for all pixels within one frame stored in an image pickup element.

2. Description of the Related Art

In an electronic endoscope apparatus, a CCD (Charge Coupled Device), e.g. a solid image pickup element reads charges stored by a photoelectric converting element so as to correspond to pixels, to form image signals (video signals). Then, provided that the electronic endoscope apparatus is of a simultaneous type, a color image is provided by color filters arranged on a top surface of the CCD so as to correspond to the pixels.

FIG. 9 shows how the color filters are arranged. As shown in this figure, for example, Mg (magenta), Cy (cyan), G (green), and Ye (yellow) filters are arranged on an image pickup surface of the CCD 1 so as to correspond to pixels. With this CCD 1, stored charges (pixel signals), corresponding to the pixels, are obtained via these color filters.

Then, according to a conventional pixel mixture reading method, stored charges corresponding to the pixels in two vertical lines of the CCD 1 are additively mixed together and then read out. For example, for charges stored by exposure during the first vertical synchronizing period of 1/60 second, video signals are read from odd-number fields, i.e. charges from lines 0 and 1 are mixed together to form mixed signals, charges from lines 2 and 3 are mixed together to form mixed signals, . . . . Then, for charges stored by exposure during the next period of 1/60 second, video signals are read from even-number fields, i.e. charges from lines 1 and 2 are mixed together to form mixed signals, charges from lines 3 and 4 are mixed together to form mixed signals, . . . .

Thus, 2-line mixed signals from the CCD 1 constitute 1-line field image. An odd-number field signal and an even-number field signal which deviate from each other by one line are alternately outputted for each exposure during a period of 1/60 second. These odd- and even-number field signals are interlaced to form one frame image. This image is displayed on a monitor as a motion picture or a still image.

However, with the electronic endoscope apparatus, as described above, an odd-number field signal and an even-number field signal which are used to form one frame image deviate from each other by 1/60 second. If during this period, movement of the endoscope itself or a subject, or the like occurs, then the quantity (resolution, color shift, or the like) of in particular a still image is degraded.

The applicant thus employed an all-image reading method of using a light blocking shutter to set a predetermined light blocking period and reading signals for all pixels obtained by the preceding same exposure. However, driving of the light blocking shutter, which sets this light blocking period, results in a mechanical (gear or the like) delay in response. That is, the light blocking period, used to read signals, requires a perfect light shading state. Accordingly, the light blocking shutter is operated slightly before the light blocking period taking its response time into account. This response operation (the operation performed before light is perfectly blocked) reduces the quantity of light during the preceding exposure. Furthermore, if an aperture mechanism is used to adjust the quantity of light emitted by a light source, the response time of the light blocking shutter varies depending on the aperture of the aperture. Disadvantageously, the insufficiency of light varies.

FIG. 10 shows the relationship between an aperture member of the aperture mechanism and the light blocking shutter, which sets a light blocking period. For example, an diaphragm blade 3 and a light blocking shutter 4 are arranged to block a beam 100 from a light source. The diaphragm blade 3 is attached so as to rotationally move clockwise around a rotating shaft 3A. The light blocking shutter 4 is attached so as to rotationally move clockwise around a rotating shaft 4A. The diaphragm blade 3 is driven so as to, for example, increase the quantity of light at a far point, while reducing the quantity of light at a near point. This provides such control as keeps the brightness of images fixed. Further, the light blocking shutter 4 is controlled so as to rotate once at a predetermined speed to perfectly block the beam 100 for a light blocking period of 1/60 second.

With this configuration, as shown in FIG. 10, much time is required before the beam 100 can be blocked. Further, the timing with which the light blocking shutter 4 shades the actual beam 100 varies depending on the set position of the diaphragm blade 3. That is, when the diaphragm blade 3 is fully open, the light blocking shutter 4 must be moved through a rotation angle α₁. When the diaphragm blade 3 is at the position indicated by the solid line in the figure, the light blocking shutter 4 must be moved through a rotation angle α₂. When the diaphragm blade 3 is at the position indicated by the alternate long and two short dashes line in the figure, the light blocking shutter 4 must be moved through a rotation angle α₃. Consequently, the response time changes to change the brightness of images.

FIG. 11 shows how optical output is if the light blocking shutter in FIG. 10 is used. The light blocking shutter 4 forms a shutter driving pulse in FIG. 11(B) in response to an O (Odd)/E (Even) signals in FIG. 11(A). That is, taking the above described mechanical response time ta, pulses are used which are inverted earlier by the time ta. In this case, light emitted by a light source device attenuates so as to draw a quadratic curve during the response time ta as shown in FIG. 11(C). Thus, an optical output Lt is a quantity of light La short. The quality of light La corresponding to this insufficiency varies depending on the response time as described above. As a result, images formed using the optical output Lt in FIG. 11(C) are not provided with a stable quantity of light and thus have a varying brightness.

Further, FIG. 11(D) shows an electronic shutter operation performed if an electronic shutter function is used. This electronic shutter function, for example, controls shutter time to Tb. Also in this case, the response time ta of the light blocking shutter 4 leads to light that is a quantity of light La short. The quantity of light La corresponding to this insufficiency varies depending on a variation in response time Ta. Consequently, the brightness of images is not kept fixed.

To avoid a variation in a decrease in the quantity of light, the applicant has proposed in Japanese Patent Laid-Open No. 11-276432 that the response speed be improved by using a liquid crystal shutter instead of the mechanical light blocking shutter 4. However, this liquid crystal shutter causes heat to be generated over time. Disadvantageously, the generation of heat hinders the liquid crystal from maintaining optical transparency and non-transparency. This degrades the shutter function.

Furthermore, with the conventional electronic endoscope apparatus, light from the light source is supplied to the leading end of the apparatus by the light guide. Thus, disadvantageously, light finally emitted from the leading end is hindered from becoming completely white light.

FIG. 12 shows how light from the light source is transmitted in the conventional apparatus. Light from a light source lamp 6 has such a light distribution that luminance is higher toward the center of the lamp. Further, light output by the light source lamp 6 is incident on a condenser lens 7 from random directions. Furthermore, light emitted from the condenser lens 7 is incident on a light guide 8 having a unique numerical aperture (NA). These and other factors may cause light in a particular wavelength band to appear at an exit facet of the light guide 8. That is, as shown in FIG. 12, for example, a red, yellow, blue, and other wavelength bands have different light emitting directions. Accordingly, light in a particular wavelength band appears markedly in emitted light. This prevents the obtainment of favorable white light suitable for observations with an endoscope.

### SUMMARY OF THE INVENTION

The present invention is provided in view of these problems. It is a first object of the present invention to provide an electronic endoscope apparatus that can set a light blocking period so as to maintain favorable responsiveness without degrading shutter functions, to provide high-quality images with a stable brightness.

It is a second object of the present invention to provide an electronic endoscope apparatus that can emit favorable white light from its leading end which light is suitable for observations with an endoscope and that can control the quantity of light at a high response speed without relying on an aperture mechanism with a mechanism structure.

To accomplish this object, the present invention provides an electronic endoscope apparatus comprising a light guide used to guide light from a light source to a leading end of an endoscope, a micromirror device having micromirrors each of which changes a polarization angle, to reflect the light source light so as to allow it to enter the light guide, a micromirror device driving circuit which drives micromirrors to a light guide non-incident angle to set a light blocking period used to read signals for all pixels stored in an image pickup device, an image pickup element which picks up an image of a subject on the basis of light emitted from the leading end, and an image pickup element driving circuit which utilizes the light blocking period to read signals for all pixels for one frame stored in the image pickup element by the same exposure.

According to the present invention, the apparatus is configured to guide the light source light to the light guide utilizing the micromirror device, which is produced by, for example, a CMOS semiconductor technology. Further, for example, a light blocking period for one field is set by driving micromirrors of the micromirror device to the light guide non-incident angle. Then, stored charges (pixel signals) are sequentially read which correspond to all pixels stored in the image pickup element during one field period preceding this light blocking period. That is, during the light blocking period, signals are read from odd-number lines. During the next period, signals are read from even-number lines. The light blocking period is set so that the signals are read from the even-number lines so as to prevent new signals from being stored.

When the light blocking period is set in this manner, the micromirror device operates very quickly without any delays in response. Favorable images with a sufficient light exposure are thus obtained. Reading signals for all pixels can be carried out only when a still image is formed. This serves to form a motion picture that faithfully reproduces the motion of a subject or the like and to obtain a still image not affected by any motions.

Further, the micromirror device driving circuit can adjust the quantity of light incident on the light guide to control the brightness of images, by changing the polarization angles in the micromirror device on the basis of an exposure control signal, e.g. controlling the number of micromirrors driven to the light guide non-incident angle. That is, the polarization angles of some of the micromirrors are driven the light guide non-incident angle on the basis of the exposure control signal. This allows the adjustment of the quantity of light output by the light source device. Thus, advantageously, the micromirror device can be used to both set the light blocking period and control exposure. Therefore, the configuration is simplified.

Furthermore, the micromirror driving device controls the micromirror device to set the light blocking period only when a still image is formed. To form a motion picture, the image pickup element driving circuit reads, for each field, image signals stored in the image pickup element without setting the light blocking period. To form a still image, the image pickup element driving circuit reads signals for all pixels stored in the image pickup element by the same exposure.

Moreover, the micromirror device driving circuit arranges the respective polarization angles of the micromirrors in a predetermined manner so that the desired white light is emitted from the leading end. The quantity of light source light can be controlled by evenly choosing from the micromirrors with the different polarization angles and driving the polarization angles of the chosen micromirrors to the light guide non-incident angle.

According to another aspect of the present invention, the desire white color is obtained by arranging the respective polarization angles of the micromirrors in a predetermined manner. This avoids biasing toward a particular wavelength band. In contrast, it is also possible to obtain white light with a particular wavelength distribution which is suitable for observing the coelom including blood and mucous membranes, most of which is red.

### BRIEF DESCRIPTION OF THE DRAWINGS

FIG. 1 is a block diagram showing a configuration of an electronic endoscope apparatus according to a first embodiment of the present invention;

FIG. 2 is a diagram schematically showing a configuration of a micromirror device according to the embodiment;

FIG. 3 is a diagram showing image data processed by a memory and a mixing circuit in FIG. 1;

FIG. 4 is a waveform diagram showing a signal reading operation performed to form a still image according to the first embodiment;

FIG. 5 is a waveform diagram showing operations performed if an all-pixel reading method is applied to a motion picture according to the first embodiment;

FIG. 6 is a block diagram schematically showing a configuration of an endoscope apparatus according to a second embodiment of the present invention;

FIG. 7 is a diagram showing how a plurality of polarization angles are arranged on the basis of a setting by a micromirror device according to the second embodiment;

FIG. 8 is a diagram showing the positions of micromirrors chosen by the micromirror device to control the quantity of light according to the second embodiment;

FIG. 9 is a diagram illustrating a configuration of color filters in a conventional CCD and a pixel mixture reading method used for this CCD;

FIG. 10 is a diagram showing a configuration of an diaphragm blade and a light blocking shutter in a conventional light source device;

FIG. 11 is a waveform diagram showing the relationship between response time and the insufficiency of light observed if the light blocking shutter in FIG. 10 is used; and

FIG. 12 is a diagram illustrating how light from a light source is transmitted in a conventional electronic endoscope apparatus.

### DETAILED DESCRIPTION OF THE PREFERRED EMBODIMENTS

First Embodiment

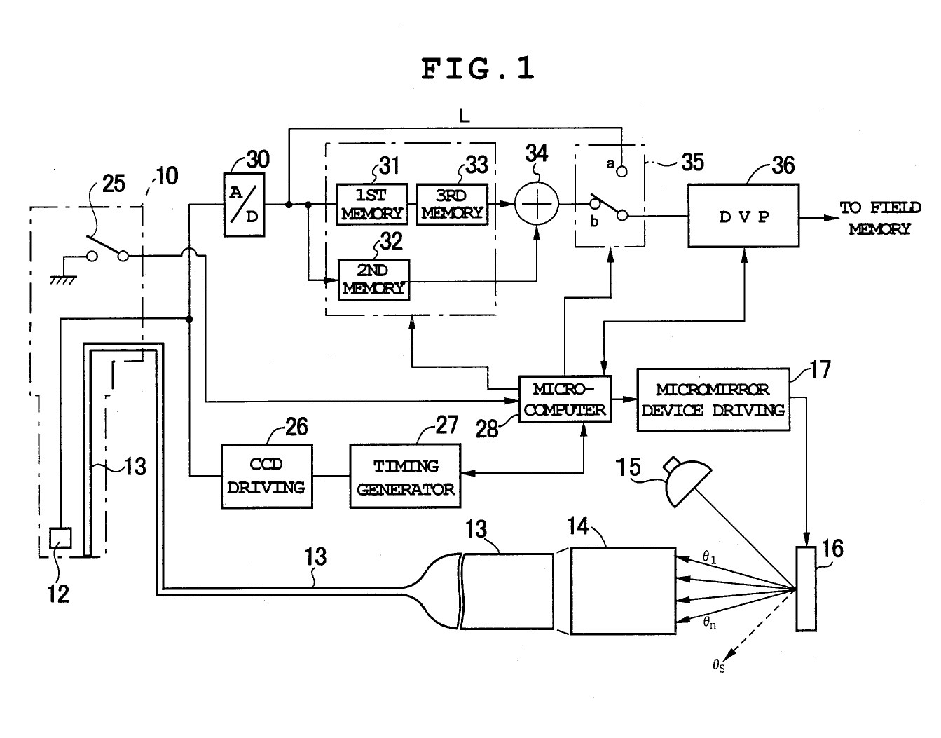

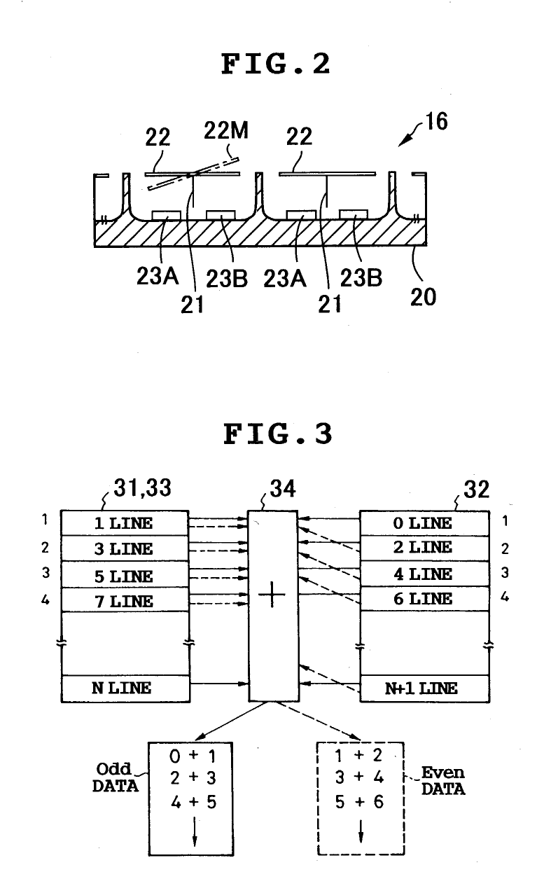

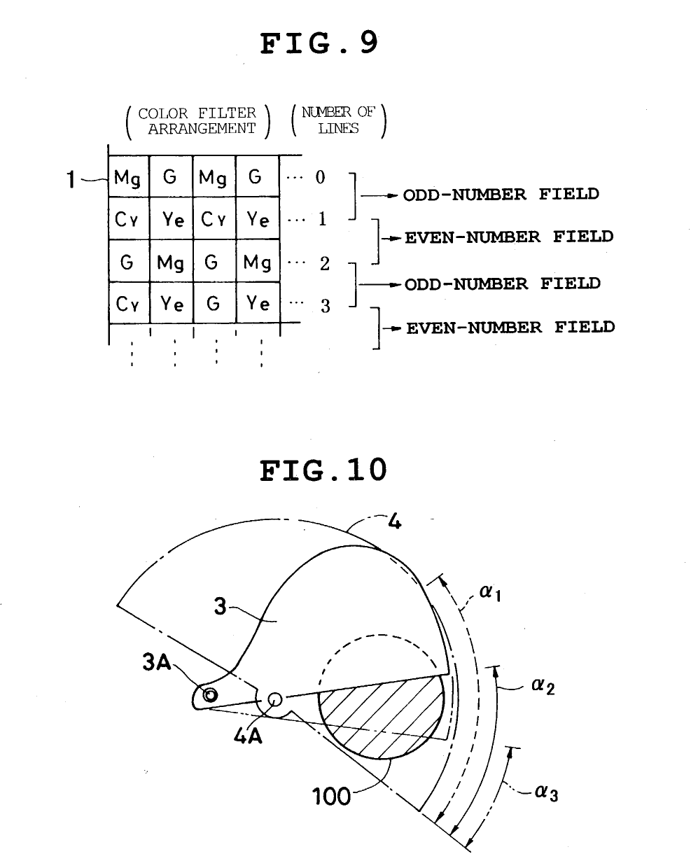

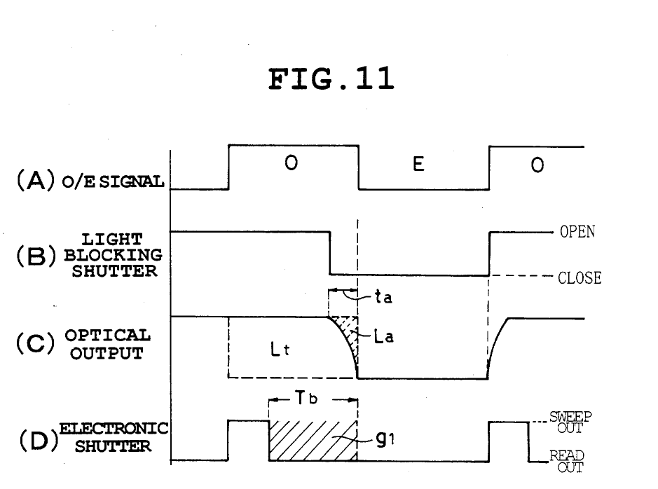

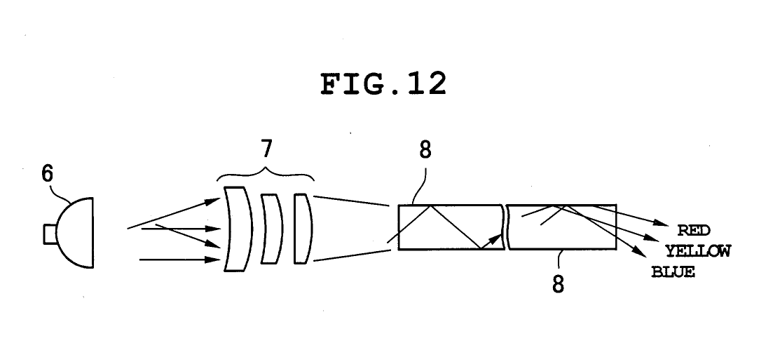

FIGS. 1 and 2 shows a configuration of an electronic endoscope apparatus according to a first embodiment. In FIG. 1, an electronic endoscope 10 is provided with a CCD 12 at its leading end which includes the color filters described in FIG. 9, and a light guide 13 extending from the leading end to a light source device. In this light source device, a condenser lens 14 is optically connected to the light guide 13. The light source device is also provided with a micromirror device 16. A micromirror device driving circuit 17 is connected to the micromirror device 16.

The micromirror device 16 is a digital micromirror device in which, for example, about five hundred thousand micromirrors are spread all over a 1.5×1.5 cm silicon memory chip through the CMOS semiconductor technology so that the polarization angle of each of these micromirrors can be controlled (for example, Japanese Patent Laid-Open Nos. 6-308397 and 6-132903).

FIG. 2 schematically shows a configuration of the micromirror device 16. In this device, for example, over 10×over 10 micron micromirrors 22 are formed on a substrate 20 via support shafts 21 so as to be freely polarized (tilted). Driving transistors (electrodes) 23A and 23B are formed below each of the micromirrors 22. The micromirror device 16 is driven by the micromirror device driving circuit 17. When the micromirror device driving circuit 17 provides a driving signal to operate, for example, a transistor 23A, the micromirror 22 is tilted as shown by a chain line 22M. In this manner, the polarization angle (tilt angle) can be arbitrarily controlled between θ1 and θn.

With this micromirror device 16, light from the light source lamp 15 can be supplied to an entrance facet of the light guide 13 via the condenser lens 14 by controlling the polarization angles of the micromirrors 22. Further, light from the light source lamp 15 can be blocked by driving the polarization angle of each of the micromirrors 22 to a non-incident angle θs (for example, 0°). At the non-incident angle θs, reflected light from the light source lamp 15 is not incident on the condenser lens 14. A light blocked state can be achieved by eliminating reflected light from all micromirrors 22.

In FIG. 1, an operation section of the endoscope 10 is provided with a freeze switch 25 used to display still images. Further, a CCD driving circuit 26 is connected to the CCD 12 to drive it. A timing generator 27 and a microcomputer 28 performing various control operations are connected to the CCD driving circuit 26. An operation signal from the freeze switch 25 is inputted to the microcomputer 28. The CCD driving circuit 26 receives an input timing signal on the basis of control provided by the microcomputer 28. It executes driving control on the basis of a CCD output pixel mixture reading method for motion pictures and an all-pixel reading method for still images.

For example, with the all-pixel reading method, the CCD driving circuit 26 supplies two types of pulses required to read stored signals for all pixels stored in the CCD 12 by the same exposure, from odd-number lines and then from even-number lines. On the basis of these pulses, such control is provided that signals are sequentially read from the odd-number lines and from the even-number lines in the CCD 12.

On the other hand, for all-pixel reading, the CCD 12 is followed, via an A/D converter 30, by a first memory 31 that stores image (signal) data from the odd-number lines, a second memory 32 that stores image data from the even-number lines, a third memory 33 for phase adjustment which stores the data from the first memory 31 as it is and which delays a read timing 1/60 second, and a still image mixing circuit 34.

That is, signals for all pixels obtained by the CCD 12 are stored in the memories 31 and 32 so that the even-number line data are stored in the memory 31, while the odd-number line data are stored in the memory 32. The odd-number line data in the first memory 31 are delayed 1/60 second so as to have the same phase as the even-number line data, stored in the second memory 32. This enables both image data to be simultaneously read. The following mixing circuit 34 can additively mix the odd-number line pixel data from the third memory 33 and the even-number line pixel data from the second memory 32 together (still image pixel mixing process).

FIG. 3 shows the contents of still image data processed by the above described memories 31 to 33 and mixing circuit 34. As shown in the right of this figure, the data from the odd-number lines (1, 3, 5, . . . lines) of the CCD 12 are stored in the first memory 31 and third memory 33. Further, as shown in the right of this figure, the data from the even lines (0, 2, 4, . . . lines) are stored in the second memory 32.

For example, the mixing circuit 34 mixes the data in the memories 33 and 32 together in terms of pixels, sequentially from above as shown by the solid or dotted line in the figure. As a result, 0 line+1 line, 2 line+3 line, 4 line+5 line, and other additive data, shown by the solid line, are output as odd field data. On the other hand, 1 line+2 line, 3 line+4 line, 5 line+6 line, and other additive data, shown by the chain line, are output as even field data.

In FIG. 1, the mixing circuit 34 is followed by an image switching circuit 35 that switches from motion picture to still image. The image switching circuit 35 has a terminal a provided with an output from the A/D converter 30 via a line L in order to form a motion picture, and a terminal b arranged opposite the terminal a and provided with an output from the mixing circuit 34. Pushing the freeze switch 25 switches the terminal a to the terminal b. A DVP (Digital Video Processor) 36 is connected to the image switching circuit 35. The DVP 36 executes various processes including gamma correction to form a signal or a luminance signal. The DVP 36 is followed by a memory that stores data from odd- and even-number fields, a D/A converter, and the like. Video signals are output to a monitor via this D/A converter.

Further, in this embodiment, the micromirror device 16 is driven to control exposure, on the basis of control provided by the microcomputer 28 and micromirror device driving circuit 17 on the basis of a luminance signal input by the DVP 36. That is, the microcomputer 28 receives a luminance signal for an image as an exposure control signal. If the luminance in the luminance signal is high, the micromirror device 16 drives micromirrors 22 the number of which corresponds to the rate of extinction, to the non-incident angle θs. If the luminance in the luminance signal is low, the micromirror device 16 returns the micromirrors 22 driven to the non-incident angle θs, to their initial polarization angles. Thus, the brightness of images is kept fixed.

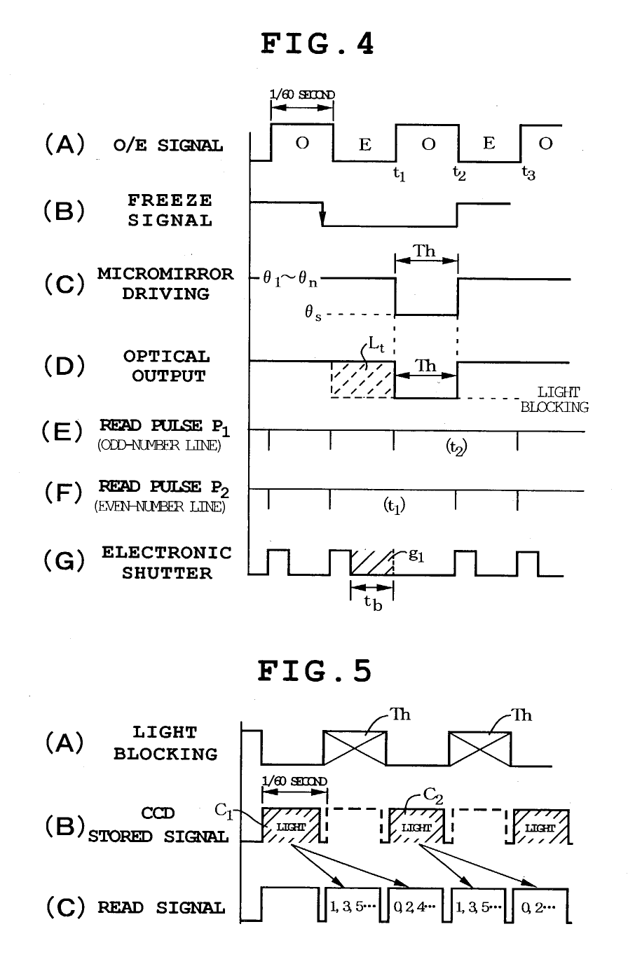

The first embodiment is configured as described above. Operations of this embodiment will be described with reference to FIG. 4. As shown in FIG. 4(A), this embodiment uses a field O (Odd)/E (Even) signal, i.e. a timing signal used to form one field image in 1/60 second. Normally, motion pictures are processed by the CCD output pixel mixture reading method. The micromirror device 16 guides reflected light from the light source lamp 15 to the leading portion of the endoscope via the condenser lens 14 and the light guide 13. Light emitted from the leading end is used to illuminate the subject.

This illumination causes charges corresponding to image light from the interior of the subject to be stored in the CCD 12. On the basis of driving pulses from the CCD driving circuit 26, the stored charges are read out so that two vertical lines are added together. A motion picture signal with pixels mixed together as shown in FIG. 9 is output. Then, this motion picture signal is fed from the A/D converter 30 to the image switching circuit 35 via the through line L. The signal is then fed to the DVP 36 via the terminal a. Operations performed by the DVP 36 and the following components are similar to those in the prior art. A motion picture is displayed on the monitor on the basis of the odd- and even-number field signals.

Further, the luminance signal obtained by the DVP 36 is fed to the microcomputer 28. The microcomputer 28 and the driving circuit 17 controls the micromirror device 16 to control the quantity of light. That is, the micromirror device 16 evenly chooses some of the lined-up micromirrors 22 according to the rate of extinction (for example, if polarization angles θ₁, θ₂, θ₃, and θ₄ are arranged in a predetermined manner, the numbers of micromirrors 22 with the respective polarization angles are evenly reduced). The micromirror device 16 drives these polarization angles to the non-incident angle θs, thus controlling the quantity of light. As result, the brightness of images is kept fixed.

On the other hand, when the freeze switch 25 of the endoscope 10 in FIG. 1 is operated to output a freeze signal as shown in FIG. 4(B), the microcomputer 28 switches the image switching circuit 35 to the terminal b to execute the all-pixel reading method. At the same time, the micromirrors 22 of the micromirror device 16 are driven to the non-incident angle θs. As shown in FIG. 4(C), the light source light is not supplied to the light guide 13 for 1/60 second after the rising of the O/E signal (t1). Light is thus blocked for a period Th. Consequently, image data for which all pixels are read correspond to charges stored in the CCD 12 by an optical output Lt during the period of 1/60 second preceding a light blocked period Th as FIG. 4(D).

That is, FIG. 4(E) indicates read pulses P1 for the odd-number lines in the left of FIG. 3. FIG. 4(F) indicates read pulses P2 for the even-number lines in the right of FIG. 3. As shown in the figure, odd-number line data and even-number line data are sequentially read according to the read pulses P1 excluding the one at t2 and the read pulses P2 excluding the one at t1. Accordingly, the odd-number line data are read during the light blocking period (t1 to t2). The even-number line data are read during the next period (t2 to t3).

FIG. 4(G) shows operations of the electronic shutter. In this case, charges stored during the rising of the pulse are swept out. Charges stored during the falling of the pulse are read out. Consequently, the abode described still image signal is obtained by exposure g1 during a shutter time tb after the charges have been swept out. The CCD driving circuit 26 reads the charges for all pixels.

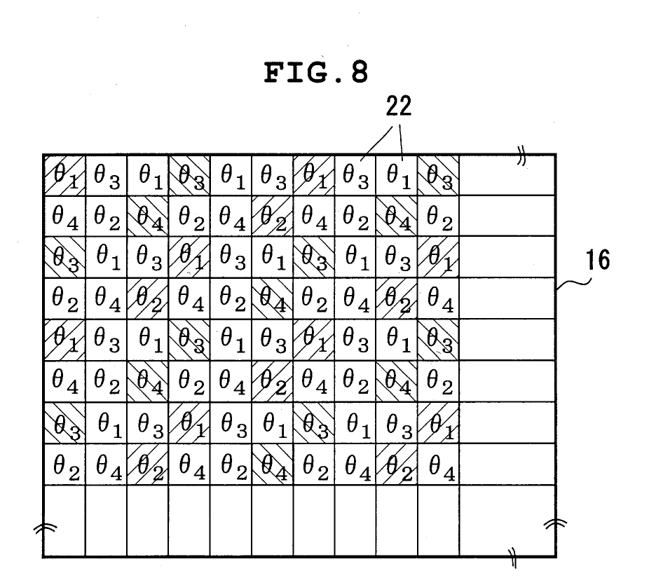

With this signal reading method, the light blocking period Th is obtained at a favorable response speed. That is, in the prior art, as described in FIG. 8, in the case of a mechanical light blocking shutter, the apparatus is operated using pulses that are a time ta earlier, the time ta corresponding the mechanical response delay of the driving section. Consequently, with the optical output Lt for still images is a quantity of light La short. However, the micromirror device 16 used in this embodiment instantaneously drives the micromirrors 22. It is thus unnecessary to take the response time ta into account. This eliminates a variation in decrease in the quantity of light, which may occur in setting the light blocking period Th. Therefore, a favorably bright still image signal is obtained whether the subject is at a far or near point. Further, operations are not affected by generated heat as in the case with the conventional liquid crystal shutter.

Then, for the still image signal obtained by the CCD 12, the odd-number line data are written in the first memory 31 and third memory 33 in FIG. 1. The even-number line data are written in the second memory 32. Then, the mixing circuit 34 mixes, in terms of pixels, pairs of data which are stored in the memories 32 and 33, respectively, and which have matching phases. As shown in FIG. 3, the mixing circuit 34 outputs 0 line+1 line, 2 line+3 line, 4 line+5 line, and other additive data as odd field data, and 1 line+2 line, 3 line+4 line, 5 line+6 line, and other additive data as even field data. Subsequently, on these field data, a still image is displayed on the monitor. Since this still image is formed of all-pixel data obtained by the same exposure, it is not virtually affected by the movement of the endoscope itself or the subject. This image thus has high quality.

FIG. 5 shows a process used to execute the all-pixel reading method on a motion picture. Not only a still image but also a motion picture can be obtained on the basis of the all-pixel reading method. That is, as shown in FIG. 5(A), the micromirror device 16 sets each vertical synchronizing period of 1/60 second alternating with a signal, as the light blocking period Th. As shown in FIGS. 5(B) and 5(C), a CCD stored signal C1 is used to read the odd-line data (1, 3, 5, . . . ) and the even-number line data (0, 2, 4, . . . ), and then a CCD stored signal C2 is used to similarly read the odd- and even-number line data. Then, for both motion picture and still image, an image is displayed which is formed of signals for all pixels for one frame obtained by the same exposure.

As described above, according to the first embodiment, the operation of setting the light blocking period is promptly performed without thermally degrading the shutter functions as in the case with the liquid crystal shutter. This embodiment also prevents a decrease in quantity of light, which may occur with the mechanical light blocking shutter. It is thus possible to provide a stably bright high-quality still image or the like which is not affected by the movement of the endoscope itself or the subject. Further, since the micromirror device controls exposure, the conventional aperture mechanism is not required. This contributes to simplifying the configuration of the light source device.

Second Embodiment

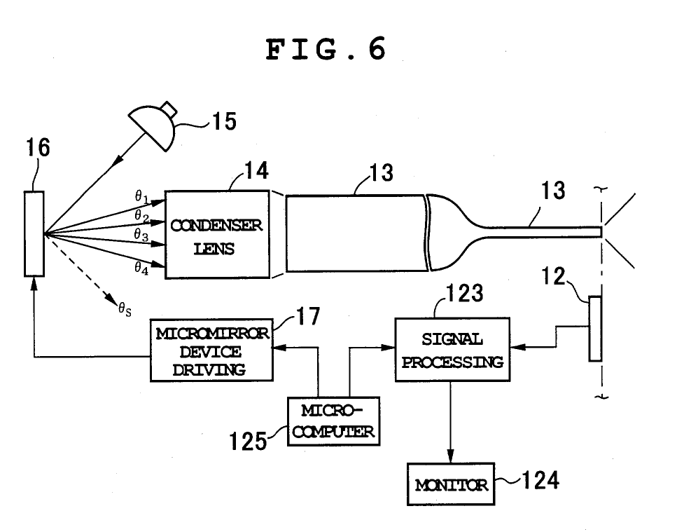

FIG. 6 schematically shows an electronic endoscope apparatus and illustrates a second embodiment. As in the case with the first embodiment, the micromirror device 16 is provided to reflect light from the light source lamp 15. The micromirror device 16 is driven by the micromirror device driving circuit 17. Further, the condenser lens 14 and the light guide 13 are arranged so as to receive reflected light from the micromirror device 16. On the other hand, the leading end of the endoscope is provided with the CCD 12. A signal processing circuit 123 subjects an output signal from the CCD 12 to a color image process or the like. A video signal formed by the signal the signal processing circuit is fed to a monitor 124.

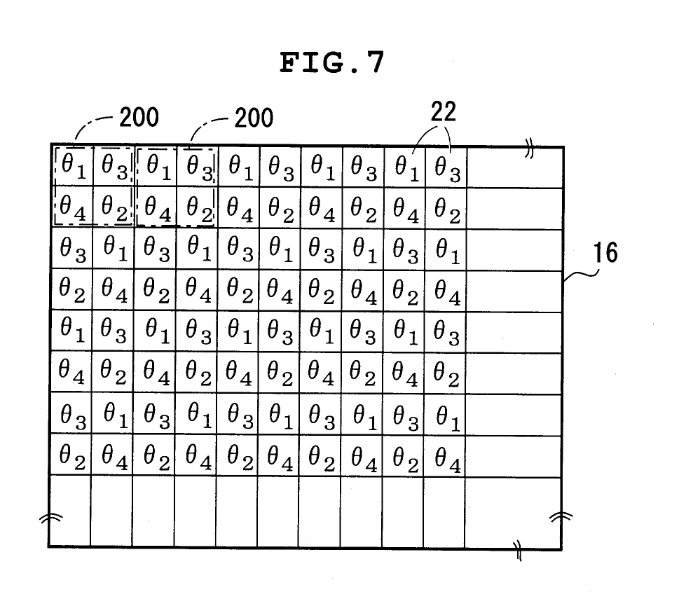

Furthermore, a microcomputer 125 is provided which integrally controls the above circuits and which provides such control as obtains white light as well as quantity-of-light control. The microcomputer 125 first controls white adjustment, described in FIG. 7. That is, the respective polarization angles of the micromirrors 22 are arranged in a predetermined manner so that the desired white light is emitted by the light guide 13. For example, the micromirrors 22 are divided into four groups with the respective polarization angles θ₁, θ₂, θ₃, and θ₄ (the number of groups is arbitrary). Then, the micromirrors 22 with these polarization angles are arranged so as to be distributed.

FIG. 7 shows how the micrometers 22 with the different polarization angles are arranged. As appreciated by examining each range labeled as 200, the polarization angles θ₁, θ₂, θ₃, and θ₄ of the micromirror 22 are arranged so as to be distributed. The polarization angles θ₁, θ₂, θ₃, and θ₄ are basically adjusted so as to prevent white light from being biased toward a particular wavelength band even if, for example, a light distribution is such that output light from the light source lamp 15 has a luminance increasing toward the center of the lamp, light reflected by the micromirror device 16 randomly enters the condenser lens 14, or the light guide 13 has a unique numerical aperture (NA). In this regard, the endoscope is used to observe the coelom including blood and mucous membranes, most of which is red. It is thus possible to obtain white light with a particular wavelength distribution which is suitable for such observations.

FIG. 8 shows how the quantity of light is controlled using such a polarization angle arrangement. For example, it is assumed that in the state in which the full quantity of light is introduced (fully open state), the quantity of light is reduced to one-third. The polarization angles of the shaded micromirrors 22 in FIG. 8 are set to the non-incident angle θs (FIG. 1) at which light does not enter the light guide 13. That is, as shown in FIG. 1, the polarization angles θ₁, θ₂, θ₃, and θ₄ set in order to form white light allow light from the light source lamp 15 to be reflected by the micromirror device 16 and then to enter the condenser lens 14. However, the micromirror device driving circuit 17 evenly chooses from the micromirrors 22 with the respective polarization angles θ₁, θ₂, θ₃, and θ₄ so that the number of micromirrors chosen equals one-third of the total number of micromirrors. The micromirror device driving circuit 17 drives these polarization angles to the value θs (for example, 0°). At the angle θs, reflected light from the light source lamp 15 is not incident on the condenser lens 14. Extinction is achieved by eliminating reflected light from the chosen mirrors 22.

The second embodiment is configured as described above. When the power supply to the apparatus is turned on, micromirror device driving circuit 17 arranges and distributes the polarization angles θ₁, θ₂, θ₃, and θ₄ of the micromirrors 22 of the micromirror device 16 as shown in FIG. 7. Then, as shown in FIG. 6, those micromirrors 22 of the micromirror device 16 which have been driven to the polarization angles θ₁, θ₂, θ₃, and θ₄, respectively, reflect the light source light. The light is then incident on the light guide 13 via the condenser lens 17. As a result, favorable white light is emitted from the leading end of the light guide 13. The CCD 12 then picks up an image of the subject, while illuminating the subject with this white light. Consequently, the monitor 124 can be used to observe the subject with natural tones.

On the other hand, on the basis of the luminance signal or the like obtained by the signal processing circuit 123 in FIG. 6, the microcomputer 125 controls the quantity of light in order to appropriately keep the image bright. The microcomputer 125 and the micromirror device driving circuit 17 provides such control that the micromirror device 16 drives the polarization angles of micromirrors 22 the number of which corresponds to the rate of extinction, to the non-incident angle θs, as described in FIG. 8.

That is, by evenly reducing the numbers of micromirrors 22 with the polarization angles θ₁, θ₂, θ₃, and θ₄ arranged in the predetermined manner, micromirrors 22 the number of which corresponds to the rate of extinction are chosen. The polarization angles of the chosen micromirrors 22 are then driven to the non-incident angle θs. The quantity of light is thus controlled. Thus, the quantity of light can be controlled while maintaining the desired white light. It is therefore possible to always obtain high-quality images.

As described above, according to the second embodiment, the quantity of light is controlled in the following manner: The micromirror device is used to arrange the respective polarization angles of the micromirrors in a predetermined manner so that the desired white light can be emitted. The micromirror device then chooses from the micromirrors with the different polarization angles arranged in the predetermined manner. The polarization angles of the chosen micromirrors are then driven to the light guide non-incident angle. Consequently, white light, which is suitable for endoscope observations, is emitted from the leading end to provide a high-quality image of the subject. Further, this micromirror device produces the following effects. It operates substantially faster than an aperture mechanism with a mechanical structure. It can promptly control the quantity of light with a high response speed. It is also unlikely to become defective.

What is claimed is:

1. An electronic endoscope apparatus comprising:
a light guide used to guide light from a light source to a leading end of an endoscope;
a micromirror device having micromirrors each of which changes a polarization angle, to reflect said light source light so as to allow the light to enter said light guide;
a micromirror device driving circuit which drives micromirrors to a light guide non-incident angle to set a light blocking period used to read signals for all pixels stored in an image pickup device;
an image pickup element which picks up an image of a subject on the basis of light emitted from said leading end; and
an image pickup element driving circuit which utilizes the light blocking period to read signals for all pixels for one frame stored in said image pickup element by the same exposure.

2. The electronic endoscope apparatus according to claim 1, wherein said micromirror device driving circuit changes the polarization angles of said micromirror devices on the basis of an exposure control signal to adjust the quantity of light incident on said light guide to control brightness of an image.

3. The electronic endoscope apparatus according to claim 1, wherein said micromirror device driving circuit controls said micromirror device so as to set the light blocking period only when a still image is formed, and
said image pickup element driving circuit provides such control that to form a motion picture, image signals stored in said image pickup element are read for each field without setting said light blocking period, and to form a still image, signals for all pixels stored in said image pickup element by the same exposure are read out.

4. The electronic endoscope apparatus according to claim 1, wherein said micromirror device driving circuit controls the quantity of light source light by arranging the respective polarization angles of said micromirrors in a predetermined manner so that desired white light is emitted from said leading end, evenly choosing from the micromirrors with the different polarization angles arranged in the predetermined manner, and driving the polarization angles of the chosen micromirrors to a light guide non-incident angle.

5. An electronic endoscope apparatus comprising:
a light guide used to guide light from a light source to a leading end of an endoscope;
a micromirror device having micromirrors each of which changes a polarization angle, to reflect said light source light so as to allow the light to enter said light guide;
a micromirror device driving circuit which drives the micromirror device to control the quantity of light source light by arranging the respective polarization angles of said micromirrors in a predetermined manner so that desired white light is emitted from said leading end, evenly choosing from the micromirrors with the different polarization angles arranged in the predetermined manner, and driving the polarization angles of the chosen micromirrors to a light guide non-incident angle; and
an image pickup element which picks up an image of a subject on the basis of light emitted from said leading end.
